(19) United States
(12) Patent Application Publication
(10) Pub. No.: US 2005/0272108 A1
(43) Pub. Date: Dec. 8, 2005

(54) Stabilized two component system for chemiluminescent assay in immunodiagnostics

(76) Inventors: Bhanu Kalra, Friendswood, TX (US); Ajay Kumar, Friendswood, TX (US)

Correspondence Address: Diagnostic Systems Laboratories, Inc.;Attn: In-house counsel, 445 Medical Center Boulevard, Webster, TX, 77598, US

(21) Appl. No.: 11/134,064

(22) Filed: May 20, 2005

Related U.S. Application Data
(60) Provisional application No. 60/572,999, filed on May 21, 2004.

(51) Int. Cl. G01N 33/53; G01N 33/537; G01N 33/543

(52) U.S. Cl. 435/7.92

(57) ABSTRACT

The present invention provides stabilized chemiluminescent formulations for use in in vitro diagnostics, including competitive as well as sandwich-type immunological assays. The stabilized assay system may be composed of two components, where the first component may contain a chemiluminescent organic compound, an enhancer, a homogenizing agent, and a suitable buffer with formulations having a pH range from about 7.2 to about 12, and optionally a solubilizing agent. The chemiluminescent system of the present invention is useful in immunoenzymatic analytical procedures, such as immunometric, competitive binding and sandwich type assays. In such immunoassays employing the chemiluminescent system of the present invention, the detectable light signal shows a proportional decay with time in the test samples and standards, so that the decay of the light emitted does not effect the concentration of the analyte measured over the entire analyte measurement range of the immunoassay. This allows accurate measurement of analyte concentrations in a test sample over extended periods of time.

### CROSS-REFERENCE TO RELATED APPLICATION

This non-provisional patent application claims benefit of provisional patent application U.S. Ser. No. 60/572,999, filed May 21, 2004, now abandoned.

### FIELD OF THE INVENTION

The present invention relates to compositions comprising stabilized two component chemiluminescent assays and methods thereto. The compositions are useful for detecting the presence of a target antigen/antibody in a sample with high sensitivity and stability.

### BACKGROUND OF THE INVENTION

Enzyme conjugates are widely used in enzyme-linked immunosorbent assays (ELISA). Most frequently, horseradish peroxidase and alkaline phosphatase enzymes are used as conjugates because of their high turnover rate, stability, ease of conjugation and relatively low cost. The sensitivity and the dynamic range of an immunoassay are very much dependent on the detection system used. The colorimetric assay is restricted to a maximum of two decades of linearity due to the physico-chemical limitation of absorbance measurement (Lambert & Beer law).

Isotopic methods have been proven to provide good sensitivity and dynamic range in immunoassays, but they are user-unfriendly and unsafe. The radiation from isotopic methods can penetrate through media and results in a short assay shelf life and disposal problems. The present invention uses chemiluminescence technology, a non-radioactive method that can be used in a tube or microtiter plate format. Chemiluminescence provides several advantages over isotopic methods, including improved sensitivity, a broad dynamic range, and rapid and consistent signals.

Horseradish peroxidase (HRP) conjugate bound to the plate in the presence of peroxide catalyzes the oxidation of diacylhydrazides to form a product in the excited state (see FIG. 1 herein). This product when decays to the ground state releases energy by emitting light. An electron transfer agent in the substrate solution enhances the light emission with increased light duration.^{1,2} The rate of signal generated and detected by a commercial luminometer should be directly proportional to the amount of HRP bound to the solid surface.

The chemiluminescence technology used in the present invention uses a system of at least two components, which may comprise a luminogenic substrate, an enhancer, and peroxide as the oxidant in a buffered solution. The chemiluminescent system of detection is based on the enhancement of peroxidase-dependent oxidation of cyclic diacylhydrazides by compounds such as substituted phenols. An intense chemiluminescent signal is obtained that is relatively stable for more than 15 minutes. This reagent composition has been optimized for sandwich/competitive binding in immunoassays for a peroxidase-based detection system on a micro plate chemiluminescence reader.

### SUMMARY OF THE INVENTION

The present invention provides stabilized chemiluminescent formulations for use in in vitro diagnostics, including competitive as well as sandwich-type immunological assays.

An embodiment of the present invention comprises a stabilized chemiluminescent assay system comprising a luminogenic substrate, an enhancer and an oxidant in a high ionic strength organic-based buffer such as barbital, tris, borate or carbonate, and a preferred pH of from about 7.2 to about 12.0.

In one embodiment, the assay system comprises two components, Component A and Component B. Component A comprises (a) at least one chemiluminescent organic compound, (b) at least one enhancer, (c) at least one homogenizing agent, and (d) at least one suitable buffer with formulations having a pH range from about 7.2 to about 12. Component B comprises at least one stabilized oxidizing agent. Component A may also comprise at least one solubilizing agent.

Another embodiment of the present invention features the use of a substrate in immunoenzymatic analytical procedures, such as immunometric, competitive binding and sandwich type assays (David et al., U.S. Pat. No. 4,486,530, hereby incorporated by reference). One embodiment includes a method for performing chemiluminescence assays which comprises: (a) mixing component A and component B, (b) providing a detection probe which reacts with the substrate to emit detectable light, and (c) detecting the relative light emitted by the interaction of probe with the substrate.

An additional embodiment provides a method of performing an immunological assay of an analyte concentration in a test sample using the chemiluminescent system of the present invention, comprising the steps of: mixing component A and component B to form a premixed solution; providing an analyte in a test sample and in a standard of known analyte concentration; providing an antibody conjugated to a detection probe which reacts with the chemiluminescent substrate to emit detectable light, wherein said antibody binds to the analyte in the test sample and in the standard; contacting said antibody separately with the analyte in the test sample and in the standard to allow binding of the antibody to the analyte in the test sample and in the standard, forming a bound test sample and a bound standard; contacting the premixed solution separately with the bound test sample and the bound standard, allowing the detection probe to react with the chemiluminescent substrate to emit detectable light; detecting the relative light emitted by the interaction of the detection probe with the chemiluminescent substrate in the bound test sample and the bound standard; and calculating the analyte concentration in the test sample based on the relative amounts of light detected in the bound test sample and the bound standard after contact with the premixed solution. The light emitted in the bound test sample and in the bound standard shows a proportional decay with time, so that the decay of the light emitted does not effect the concentration of the analyte measured over the entire analyte measurement range of the immunoassay. This allows accurate measurement of analyte concentrations in a test sample over extended periods of time using the chemiluminescent system of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specific embodiments presented herein.

FIG. 1 depicts the catalysis by horseradish peroxidase (HRP) conjugate in the presence of peroxide of the oxidation of diacylhydrazides to form a product in the excited state. This product releases energy by emitting light when it decays to the ground state. An electron transfer agent in the substrate solution enhances the light emission with increased light duration.^{1,2}

FIG. 2 shows the concentration of the HRP versus RLU response in micro titer plates.

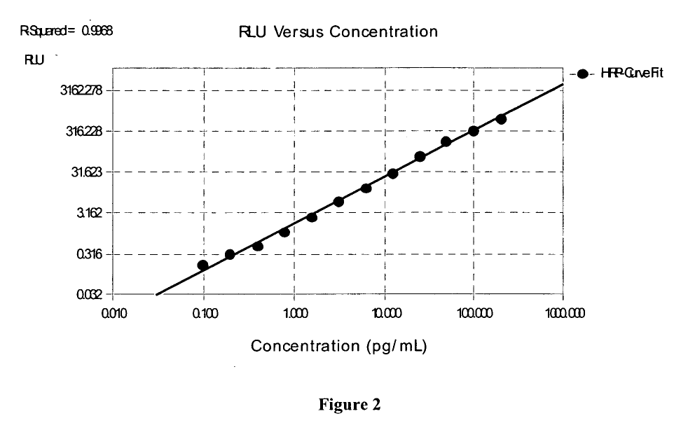

FIG. 3 shows the concentration of the HRP versus OD response in micro titer plates.

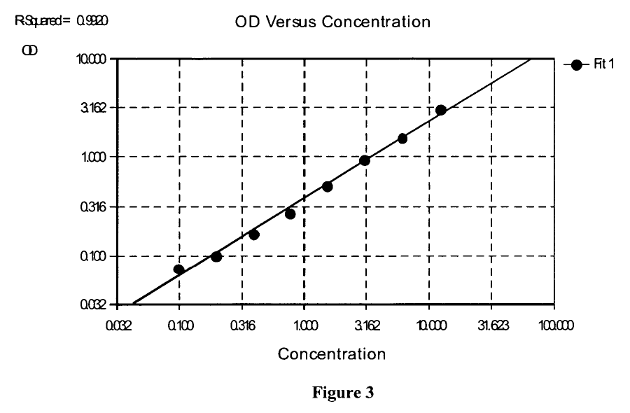

FIG. 4 shows the sigmoid curve for unconjugated Estriol (representative example for competitive binding assay) using the present invention.

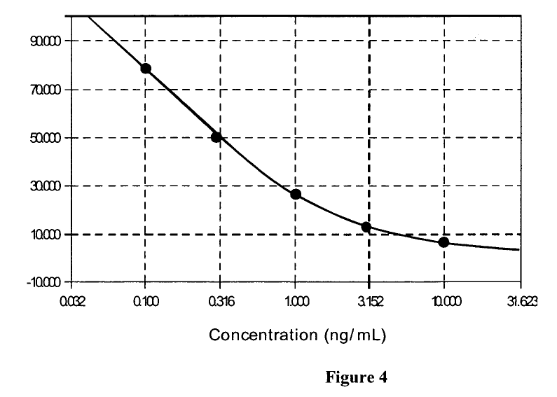

FIG. 5 shows the linear regression curve for Inhibin-A (representative example for sandwich assay) using the present invention.

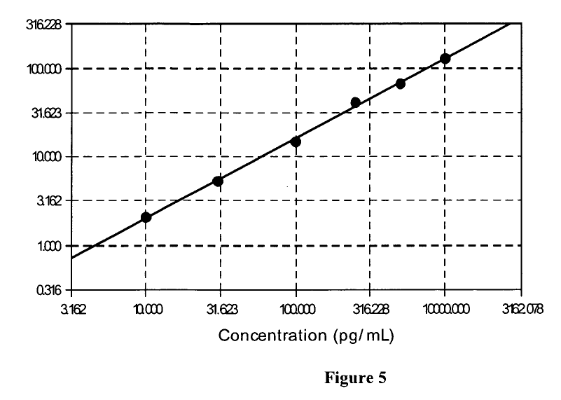

### DETAILED DESCRIPTION OF THE INVENTION

The present invention provides stabilized two component chemiluminescent formulations for use as substrates in in vitro diagnostics, including competitive as well as sandwich-type immunologicalassays.

An embodiment of the present invention comprises a stabilized two-component chemiluminescent assay system containing a luminogenic substrate, an enhancer and an oxidant in a high ionic strength organic-based buffer such as barbital, borate, tris or carbonate, and a preferred pH of from about 7.2 to about 12.0.

In one embodiment, the assay system comprises two components, Component A and Component B. In one embodiment, Component A comprises (a) at least one chemiluminescent organic compound, (b) at least one enhancer, (c) at least one homogenizing agent, and (d) at least one suitable buffer with formulations having a pH range from about 7.2 to about 12. Component B comprises at least one stabilized oxidizing agent. Representative examples of an oxidizing agent include hydrogen peroxide, urea hydrogen peroxide, a perborate salt and mixtures thereof. Representative examples of the chemiluminescent compound include luminol, isoluminol, phenyl-10-methylacridane-9-carboxylate, 2,4,6-trichlorophenyl-1-0-methylacridane-9-carboxylate, acridane, pyrogallol, phloroglucinol, resorcinol, and their salts and mixtures thereof.

Additional representative examples of a chemiluminescent compound include resorcinol, pyrogallol, phloroglucinol, purpurogallin, aminoaryl cyclic diacylhydrazide or the salts thereof, wherein the aryl group maybe phenyl, substituted phenyl, naphthyl, substituted naphthyl, anthryl or substituted anthryl; hydroxyaryl cyclic diacylhydrazide, where the aryl group is phenyl, substituted phenyl, naphthyl, substituted naphthyl, anthryl or substituted anthryl; pyridopyridazine derivatives; acridanes, substituted acridanes, such as 10,10′-dimethy-9,9′-biacridane, 9-benzylidene-10-methylacridane, substituted-9-benzylidene-10-mrthylacridane, N-methylacridane or substituted N-methylacridane, 9-benzylacridane, substituted-9-benzylacridane, 9-benzyl-N-methylacridane, substituted-9-benzyl-N-methylacridane, N-alkylacridane-9-carboxylic acid, an ester or thioester thereof, indole-3-acetic acid, an ester or thioester thereof, N-methylindole-3-acetic acid, an ester thereof, phenyl or substituted phenyl-2-(6′-hydroxy-2-benzothiazolyl-.DELTA.sup..2-thiazoline-4-carboxylate, methyl 2-(6′-hydroxy-2′-benzothiazolyl)-.DELTA.sup..2-thiazoline-4-carboxylate, 2-(6′-hydroxy-2′-benzothiazolyl)-.DELTA..sup.2-t-hiazoline acetic acid or an ester thereof, 2-(4′-hydroxyphenyl) thiazole-4-carboxylic acid hydrazide, 2-(6′-hydroxy-2′-benzothiazolyl) thiazole-4-carboxylic acid hydrazide, 9-acridanecarboxylic acid hydrazide, substituted 9-acridanecarboxylic acid hydrazide, N-alkyl-9-acridanecarboxylic acid hydrazide, substituted N-alkyl-9-acridanecarboxylic acid hydrazide, o-hydroxybenzoic acid hydrazide, o-aminobenzoic acid hydrazide, m-hydroxybenzoic acid hydrazide, 2-hydroxy-3-naphthoic acid hydrazide, 2-amino-3-naphthoic acid hydrazide, 1-hydroxy-2-anthroic acid hydrazide, D-luciferin-O-sulfate, D-luciferin-O-phosphate, luciferins isolated from Pholas dactius, the firefly Photinus pyrali or Cypridina, as well as mixtures thereof. Additional examples include luminol, isoluminol, phenyl-10-methylacridane-9-carboxylate, 2,4,6-trichlorophenyl-1-0-methylacridane-9-carboxylate, acridane, pyrogallol, phloroglucinol, resorcinol, and mixtures thereof.

Representative examples of a homogenizing agent include ionic, nonionic surfactions, proteins, carbohydrates and natural as well as synthetic polymers. Nonionic surfactants include glycerol, propylene glycol, as well Tween 20, Tween 40, Tween 60, Tween 80, Tween 85, Triton X-100, Triton X-100 (reduced), Triton N-101, Triton N-101 (reduced), Triton X-114, Triton X-114 (reduced), Triton X-405, Triton X-405 (reduced), Brij 35 and the like; other useful agents include ionic surfactants such as lauryl sulfate, domiphen bromide, cetyltrimethyl ammonium bromide, cetyltrimethyl ammonium chloride, cetyldimethylethyl ammonium bromide (CTAB); proteins, such as gelatins, bacitracin, BSA, KLH, HSA, Trypsin inhibitor; polymers such as polyvinyl alcohols, polylysines; carbohydrates such as hydroxyethyl cellulose, hydroxypropyl cellulose, carboxymethyl cellulose, as well as inorganic pyrophosphates, ethylenediaminetetraacetic acid (EDTA), diethylenetriaminepentaacetic acid, ethylene-bis(oxyethylenenitrilo) tetraacetic acid, and other related macromolecules as well as any other compounds capable of stabilizing as well as homogenizing the chemiluminescent compound in the formulation and mixtutes thereof. Representative examples of an enhancer include halogenated phenols, such as p-iodophenol, p-bromophenol, p-chlorophenol, 4-bromo-2-chlorophenol, 3,4-dichlorophenol, alkylated phenols, such as 4-methylphenol and, 4-tert-butylphenol, 3-(4-hydroxyphenyl) propionate and the like, 4-benzylphenol, 4-(2′,4′-dinitrostyryl) phenol, 2,4-dichlorophenol, p-hydroxycinnamic acid, p-fluorocinnamic acid, p-nitroicinnamic acid, p-aminocinnamic acid, m-hydroxycinnamic acid, o-hydroxycinnamic acid, 4-phenoxyphenol, 4-(4-hydroxyphenoxy) phenol, p-phenylphenol, 2-chloro-4-phenylphenol, 4′-(4′-hydroxyphenyl) benzophenone, 4-(phenylazo) phenol, 4-(2′-carboxyphenylaza) phenol, 1,6-dibromonaphtho-2-ol, 1-bromonaphtho-2-ol, 2-naphthol, 6-bromonaphth-2-ol, 6-hydroxybenzothiazole, 2-amino-6 -hydroxybenzothiazol-e, 2,6-dihydroxybenzothiazole, 2-cyano-6-hydroxybenzothiazole, dehydroluciferin, firefly luciferin, phenolindophenol, 2,6-dichlorophenolindophenol, 2,6-dichlorophenol-o-cresol, phenolindoaniline, N-alkylphenoxazine or substituted N-alkylphenoxazine, N-alkylphenothiazine or substituted N-alkylphenothiazine, N-alkylpyrimidyl-phenoxazine or substituted N-alkylpyrimidylphenoxazine, N-alkylpyridylphenoxazine, 2-hydroxy-9-fluorenone or substituted 2-hydroxy-9-fluorenone, 6-hydroxybenzoxazole or substituted 6-hydroxybenzoxazole. Still other useful compounds include a protected enhancer that can be cleaved by the enzyme such as p-phenylphenol phosphate or p-iodophenol phosphate or other phenolic phosphates having other enzyme cleavable groups, as well as p-phenylene diamine and tetramethyl benzidine. Other useful enhancers include fluorescein, such as 5-(n-tetradecanyl) amino fluorescein and the like.

In another embodiment, component A may also comprise at least one solubilizing agent. The solubilizing agent may be present in amounts of from about 0.05% to about 10%, by volume, based on the total volume. Representative examples of a solubilizing agent include dimethyl formamide, dimethyl sulfoxide, Tetrahydrofuran, dioxane, alcohols and mixtures thereof.

In another embodiment, the component A formulation comprises (a) from about 0.01% to about 10% based on weight, of the chemiluminescent compound, (b) from about 0.01% to about 10% based on weight, of the enhancer, and (c) from about 0.01% to about 30% based on weight, of the homogenizer.

Yet another embodiment of the present invention provides a method for performing chemiluminescence assays, comprising the steps of: (a) mixing a component A and a component B, (b) providing a detection probe which reacts with a substrate to emit detectable light, and (c) detecting the relative light emitted by the interaction of a probe with the substrate. The probe may comprise an enzyme, haemoglobin, protohemin, cytochrome C or related biomimetic models, or horseradish peroxidase, soyabean peroxidase, xanthine oxidase, catalase, laccase as well as mixtures thereof.

Another embodiment of the present invention features the use of a substrate in immunoenzymatic analytical procedures, such as immunometric, competitive binding and sandwich type assays (David et al., U.S. Pat. No. 4,486,530). One such embodiment includes a method for performing chemiluminescence assays which comprises the steps of: (a) mixing a component A and a component B, (b) providing a detection probe which reacts with a substrate to emit detectable light, and (c) detecting the relative light emitted by the interaction of the detection probe with the substrate. The probe may comprise an enzyme, haemoglobin, protohemin, cytochrome C or related biomimetic models. Examples of a suitable enzyme include horseradish peroxidase, soyabean peroxidase, xanthine oxidase, and catalase, as well as mixtures thereof. The peroxidase based chemiluminescent substrate system can be adapted for use in wide variety of assays to detect an analyte, wherein a specific binding pair ligand is coupled with an enzymatic or avidin tracer. As used herein, the analyte is a specific binding material whose presence or amount is to be determined. The analyte can be antigens, haptens, antibodies, steroids, glycosylated proteins, recombinant proteins, carbohydrates, oligonucleotides, or Fab of proteins. Still another embodiment of the present invention comprises a two component detection system for chemiluminescent assays, comprising a component A which further comprises: (a) an organic chemiluminescent compound, (b) an enhancer, (c) a solubilizing solvent, (d) a homogenizing agent and (e) a buffer of a pH ranging from about 6.0 to about 12.0; and a component B further comprising a stabilized oxidant.

The reagent compositions and methods described herein have been optimized for sandwich and competitive binding immunoassays for different panels/markers including fetal well being, growth factors, androgens, estrogens, thyroid, infectious disease, bone, and metabolism, as well as diabetes, cancer and cardiac markers. The stabilized two components of this formulation have also been optimized for small peptide molecules as well as infectious disease kits, for peroxidase based detection systems using a chemiluminescence reader.

An additional embodiment of the present invention provides a method of performing an immunological assay of an analyte concentration in a test sample using the system of claim 5, comprising the steps of: mixing component A and component B to form a premixed solution; providing an analyte in a test sample and in a standard of known analyte concentration; providing an antibody conjugated to a detection probe which reacts with the chemiluminescent substrate to emit detectable light, wherein said antibody binds to the analyte in the test sample and in the standard; contacting said antibody separately with the analyte in the test sample and in the standard to allow binding of the antibody to the analyte in the test sample and in the standard, forming a bound test sample and a bound standard; contacting the premixed solution separately with the bound test sample and the bound standard, allowing the detection probe to react with the chemiluminescent substrate to emit detectable light; and detecting the relative light emitted by the interaction of the detection probe with the chemiluminescent substrate in the bound test sample and the bound standard; and calculating the analyte concentration in the test sample based on the relative amounts of light detected in the bound test sample and the bound standard after contact with the premixed solution.

An advantage of the present invention is that the light emitted in the bound test sample and in the bound standard shows a proportional decay with time, wherein the decay of the light emitted does not effect the concentration of the analyte measured over the entire analyte measurement range of the immunoassay. As a result, the analyte may be measured over an extended period of time after contact with the premixed solution, including a time period of from about one (1) to twenty (20) minutes after contacting the premixed solution with the bound test sample and the bound standard.

The following examples are given for the purpose of illustrating various embodiments of the invention and are not meant to limit the present invention in any fashion.

### EXAMPLE 1

Reagents

The substrate solution is a two-component system. Component A comprises a buffered solution containing diacylhydrazide derivatives, an enhancer, and at least one homogenizing agent. Substrate B comprises a solution containing at least one stabilized peroxide derivative.

To Prepare Component A:

### Example 1 A

To A 100 ml solution of borate buffer at pH 8.55, 0.1% BSA was added in a light-sensitive container. 2.4 mM of 3-aminophthalazide was added to the buffer, followed by the addition of 1.2 mM of p-iodophenol solution in DMSO to the buffer. Then the solution was stirred and filtered through a 0.2 μm filter and stored at 2-8° C.

### Example 1 B

To A 100 ml solution of borate buffer at pH 8.52, 0.1% PVA was added in a light-sensitive container. 1 mM of 3-aminophthalazide was added to the buffer, followed by the addition of 0.5 mM of p-iodophenol solution in DMSO to the buffer. Then the solution was stirred and filtered through a 0.2 μm filter and stored at 2-8° C.

### Example 1 C

To A 100 ml solution of borate buffer at pH 8.5, 3% of Triton X-100 was added in a light-sensitive container. 2.5 mM of 3-aminophthalazide was added to the buffer, followed by the addition of 3 mM of p-phenylphenol solution in DMSO to the buffer. Then the solution was stirred and filtered through a 0.2 μm filter and stored at 2-8° C.

### Example 1 D

To A 100 ml solution of borate buffer at pH 8.52, 0.2% of Brij-35 was added in a light-sensitive container. 2.5 mM of 3-aminophthalazide was added to the buffer, followed by the addition of 3 mM of p-phenylphenol solution in DMSO to the buffer. Then the solution was stirred and filtered through a 0.2 μm filter and stored at 2-8° C.

### Example 1 E

To A 100 ml solution of borate buffer at pH 8.5, 3% of Triton X-100 was added in a light-sensitive container. 2.5 mM of 3-aminophthalazide was added to the buffer, followed by the addition of3 mM of p-phenylphenol solution in DMSO to the buffer. 0.01% Sodium Azide was added to the solution. Then the solution was stirred and filtered through a 0.2 μm filter and stored at 2-8° C.

To prepare Component B: diluted H₂O₂ solution.

### EXAMPLE 2

Preparation of Working Solution

### Example 2A

The working solution was prepared by mixing 160 μl of substrate B in 100 mL of substrate A. The two components should be mixed thoroughly by gentle inversion at least 30 minutes at room temperature prior to use.

### Example 2B

The working solution was prepared by mixing 40 μl of substrate B in 100 mL of substrate A. The two components should be mixed thoroughly by gentle inversion at least 30 minutes at room temperature prior to use.

### Example 2 C

The working solution was prepared by mixing 100 μl of substrate B in 100 mL of substrate A. The two components should be mixed thoroughly by gentle inversion at least 30 minutes at room temperature prior to use. This premixed substrate solution is stable for at least one week at 2-8° C. The working solution should be brought to room temperature before use.

### Example 2 D

The working solution was prepared by mixing 100 μl of substrate B in 100 mL of substrate A. The two components should be mixed thoroughly by gentle inversion at least 30 minutes at room temperature prior to use. This premixed substrate solution is stable for at least one week at 2-8° C. The working solution should be brought to room temperature before use.

### Example 2E

The working solution was prepared by mixing 100 μl of substrate B in 100 mL of substrate A. The two components should be mixed thoroughly by gentle inversion at least 30 minutes at room temperature prior to use. This premixed substrate solution is stable for at least one week at 2-8° C. The working solution should be brought to room temperature before use.

### EXAMPLE 3

Stability and Storage

The component reagents are stable for at least 12 months when stored at 2-8° C. in dark. The premixed component solutions are stable for at least 1 week at 2-8° C. The component reagents are prepared as sterile solutions and do not contain any preservatives. Contamination during storage and use should be avoided.

### EXAMPLE 4

Equipment

The chemiluminescence substrate of the present invention was evaluated for a variety of Diagnostic Systems Laboratories, Inc. (DSL) sandwich/competitive assays, using commercially available microplate luminometers (Centro from Berthold, Orian from Berthold Detection, MGM instruments, DSL's LR-100, Genious from Tecan and MLX from Dynex Technologies). All these instruments used photo multiplier tube technology to quantitate the luminescence generated in the wells. Most of these luminometers have a dynamic range between 10 and 2×10⁶ relative light unit/sec (RLU/sec).

### EXAMPLE 5

Results

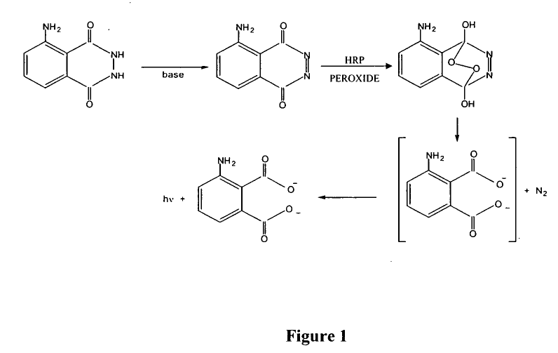

The chemiluminescence substrate of the present invention was evaluated in liquid and on coated plates using horseradish peroxidase. Non specific binding (NSB) of the substrate on a white plate was 40-800 RLU/10 msec, and the NSB on a goat anti rabbit antibody coated plate was 1000-1800 RLU/10 msec. The substrate when added to 10 μl of 0-200 Units/ml HRP solutions in micro titer wells resulted in a signal output of 1736 to 6354519 RLU/10 msec. Noise to signal ratio calculated for the above experiment was less then 0.027%. The RLU response to the concentration of the HRP is plotted in Figure-1. The log-log linear regression calculated for the above curve was >0.996 and had 3.66 decades of linearity. The slope of the curve in FIG. 1 with time was studied and was observed to be <2% change over a period of 20 minutes. Sample correlation on DSL's sandwich/competitive assays has been performed over a window of 0-15 min, and the observed coefficient of variability (CV) was less than 3%, which suggests that the plate after substrate addition could be read for at least 15 minutes with minimal variation. A comparative study with commercial TMB was also performed using 10 μl of 0-200 ng/ml HRP solutions in micro titer wells. It was observed that the OD generated was linear up to 0.195-12.5 Units/ml HRP solutions and had a signal output of 0.101 to 3.06 OD. The noise to signal ratio calculated for the above experiment was greater than 3.3%, and the sensitivity of the chemiluminescence substrate was more than 100-fold higher than TMB in colorimetric assays. The linearity range of HRP detection by the chemiluminescence substrate was >16 times that of TMB. As a result, we were able to easily replace the colorimetric substrate TMB in all ELISA/EIA assays with the chemiluminescence substrate of the present invention. Use of the chemiluminescence substrate improved the sensitivity and kinetics of most of the DSL ELISA/EIA assays.

### EXAMPLE 5

Assay of Cortisol in Human Serum/Saliva

10 μL of each Standard, Control and unknown to the appropriate wells (coated with GARG Ab) with 50 μL of the Biotin Conjugate Solution to each well and 100 μL of the Antiserum. The wells are incubated for 30 minutes at 22-28° C., followed by aspiration and washing each well five times with the Wash Solution using an automatic microplate washer. 100 μL of the streptavidin-enzyme conjugate solution was added and incubated for 15 minutes at 22-28° C. it was aspirated and washed five times with the wash solution using an automatic microplate washer. 100 μL of the substrate solution was added and was read using a microplate luminometer. The responses observed were as shown in Table 1.

TABLE 1

| | WELL | | |
|---|---|---|---|
| WELL | CONTENTS | MEAN | CONC. |
| NO. | STANDARDS | RLU × 10⁴ | (μg/dL) |
| A1, B1 | A | 71.34 | 0 |
| C1, D1 | B | 67.45 | 0.05 |
| E1, F1 | C | 58.45 | 0.2 |
| G1, H1 | D | 40.48 | 0.75 |
| A2, B2 | E | 30.19 | 1.5 |
| C2, D2 | F | 16.14 | 5.0 |
| E2, F2 | G | 7.48 | 15.0 |
| G2, H2 | H | 2.88 | 60 |

### EXAMPLE 6

Assay of Unconjugated Estriol in Human Serum

25 μL of each Standard, Control and unknown to the appropriate wells (coated with GARG Ab) with 100 μL of the Biotin Conjugate Solution to each well and 100 μL of the antiserum. It was then incubated the wells for 30 minutes at 22-28° C., followed by aspiration and washing each well five times with the Wash Solution using an automatic microplate washer. 100 μL of the Streptavidin-Enzyme Conjugate Solution was added and incubated for minutes at 22-28° C. It was aspirated and washed five times with the wash solution using an automatic microplate washer. 100 μL of the substrates Solution was added and was read using a microplate luminometer. The responses observed were as shown in Table 2.

TABLE 2

| | WELL | | |
|---|---|---|---|
| WELL | CONTENTS | MEAN | CONC. |
| NO. | STANDARDS | RLU × 10⁴ | (ng/dL) |
| A1, A2 | A | 97.625 | 0 |
| B1, B2 | B | 72.08 | 0.1 |
| C1, C2 | C | 48.105 | 0.3 |
| D1, D2 | D | 25.008 | 1 |
| E1, E2 | E | 12.356 | 3 |
| F1, F2 | F | 5.677 | 10 |

### EXAMPLE 7

Assay of Inhibin-A in Human Serum

50 μL each Standard, Control and unknown were added into the appropriate wells (coated with anti-inhibin-A antibody) followed by addition of 25 μL of Buffer A an 25 μL of buffer B. The wells were incubated for 90 min at room temperature. After 90 min., the wells were washed eight times with the wash Solution using an automatic microplate washer. 100 μL of the Antibody-biotin Conjugate Solution was added to each well using a semi-automatic dispenser followed by incubation of wells for 60 min at room temperature. After 60 min. the wells were aspirated and washed five times with the wash solution using an automatic microplate washer. 100 μL of streptavidin-HRP was added to the wells and the wells were incubated for 30 min at room temperature. After 30 min. the wells were aspirated and washed five times with the wash solution using an automatic microplate washer. 100 μL of the substrates Solution was added and the plate was read the plate within 12 minutes of the substrate addition using a microplate luminometer. The responses observed were as shown in Table 3:

TABLE 3

| | WELL | | |
|---|---|---|---|
| WELL | CONTENTS | MEAN | CONC. |
| NO. | STANDARDS | RLU × 10⁴ | (pg/mL) |
| A1, A2 | A | 0.3892 | 0.0 |
| | | (Blank) | |
| B1, B2 | B | 2.1169 | 10 |
| C1, C2 | C | 5.4525 | 30 |
| D1, D2 | D | 15.4072 | 100 |
| E1, E2: | E | 40.9680 | 250 |
| F1, F2 | F | 67.8992 | 500 |
| G1, G2 | G | 134.4084 | 1000 |

### EXAMPLE 8

Assay of Pregnancy-Associated Plasma Protein-A (PAPP-A) in Human Serum

10 μL each Standard, Control and unknown were added into the appropriate wells (coated with anti-PAPP-A antibody) followed by addition of 100 μL of conjugate in assay buffer. The wells were incubated for 90 min at room temperature. After 90 min., the wells were washed eight times with the wash Solution using an automatic microplate washer. 100 μL of the substrates Solution was added and the plate was read the plate within 12 minutes of the substrate addition using a microplate luminometer. The responses observed were as below in Table 4:

TABLE 4

| | WELL | | |
|---|---|---|---|
| WELL | CONTENTS | MEAN | CONC. |
| NO. | STANDARDS | RLU × 10⁴ | (mlU/mL) |
| A1, A2 | A | 0.0557 | 0.0 |
| | | (Blank) | |
| B1, B2 | B | 1.1093 | 0.1 |
| C1, C2 | C | 3.5124 | 0.3 |
| D1, D2 | D | 13.591 | 1.2 |
| E1, E2 | E | 37.551 | 3.0 |
| F1, F2 | F | 116.338 | 7.5 |
| G1, G2 | G | 204.577 | 15 |

### EXAMPLE 9

A proportional decay with time on standard and sample is observed. The decay in the signal does not effect the concentration of the analyte in the entire range of immunoassay. This enables to measure the RLUs over a extended time period as demonstrated in Table 5 with competitive Testosterone immunoassay.

This tolerance of the substrate enables manual user on plate format to use the immunoassay with producible results within the time period specified for each analyte (ideally 1-20 min.)

TABLE 5
Testosterone (RLU/s);

| | 1 min | 4 min | 8 min | 12 min |
|---|---|---|---|---|
| 0 | 136432 | 95045 | 76987 | 71365 |
| 0.1 | 104875 | 73503 | 59857 | 55457 |
| 0.5 | 62726 | 45391 | 35967 | 33923 |
| 2.5 | 19032 | 14472 | 12336 | 11948 |
| 5 | 8441 | 6706 | 6095 | 5872 |
| 10 | 4131 | 3294 | 3142 | 3290 |
| 25 | 1528 | 1393 | 1291 | 1391 |

| Sample | ng/mL | | | | Mean | SD | % CV |
|---|---|---|---|---|---|---|---|
| 1 | 0.687 | 0.71 | 0.67 | 0.66 | 0.68 | 0.023 | 3.4 |
| 2 | 5.27 | 5.51 | 5.48 | 5.49 | 5.43 | 0.112 | 2.06 |
| 3 | 2.88 | 2.96 | 2.98 | 3 | 2.95 | 0.053 | 1.8 |
| 4 | 2.46 | 2.56 | 2.6 | 2.58 | 2.55 | 0.062 | 2.4 |
| 5 | 2.09 | 2.08 | 2.1 | 2.17 | 2.09 | 0.062 | 2.9 |
| 6 | 1.97 | 2 | 2 | 1.99 | 1.99 | 0.014 | 0.7 |
| 7 | 3.29 | 3.3 | 3.27 | 3.36 | 3.3 | 0.038 | 1.17 |
| 8 | 1.2 | 1.27 | 1.26 | 1.27 | 1.25 | 0.033 | 2.7 |
| 9 | 4.4 | 4.48 | 4.55 | 4.63 | 4.5 | 0.098 | 2.2 |
| 10 | 3.27 | 3.4 | 3.33 | 3.3 | 3.33 | 0.056 | 1.67 |
| 11 | 0.26 | 0.26 | 0.24 | 0.23 | 0.247 | 0.015 | 6 |
| 12 | 2.42 | 2.45 | 2.45 | 2.49 | 2.64 | 0.046 | 1.79 |
| 13 | 2.58 | 2.69 | 2.64 | 2.66 | 2.64 | 0.046 | 1.76 |

Mean % CV (SD) = 2.4 +/− 1.32%

### References

1. Thorpe, G. H. G. & Kricka L. J. Methods Enzymol. 1986, 133, 331
2. Thorpe, G. H. G., Kricka L. J, Moseley S. B. & Whitehead T. P. Clin. Chem. 1985, 31/8 1335.

1. A stabilized system for use in chemiluminescent immunological assays, comprising a first component and a second component, wherein the first component further comprises a chemiluminescent organic compound, an enhancer, a homogenizing agent, and a buffer, and the second component further comprises an oxidizing agent.

2. The system of claim 1, wherein the first component further comprises a solubilizing agent.

3. The system of claim 1, wherein the first component and second component are stable for at least twelve (12) months when stored at a temperature of from about 2 to about 8 degrees Celsius and when shielded from light exposure.

4. The system of claim 1, wherein the first component and the second component are stable when mixed together for at least seven (7) days when stored at a temperature of from about 2 to about 8 degrees Celsius.

5. A stabilizedsystem for use in chemiluminescent assays, comprising a Component A and a Component B, wherein Component A comprises (a) at least one chemiluminescent organic compound, (b) at least one enhancer, (c) at least one homogenizing agent, and (d) at least one suitable buffer with formulations having a pH range from about 7.2 to about 12, and wherein component B comprises at least one stabilized oxidizing agent.

6. The system of claim 5, wherein the chemiluminescent organic compound is selected from the group consisting of resorcinol, pyrogallol, phloroglucinol, purpurogallin, aminoaryl cyclic diacylhydrazide or the salts thereof, and wherein the aryl group is selected from the group consisting of phenyl, substituted phenyl, naphthyl, substituted naphthyl, anthryl or substituted anthryl; hydroxyaryl cyclic diacylhydrazide, wherein the aryl group is selected from the group consisting of phenyl, substituted phenyl, naphthyl, substituted naphthyl, anthryl or substituted anthryl; pyridopyridazine derivatives, acridanes, substituted acridanes, 10,10′-dimethy-9,9′-biacridane, 9-benzylidene-10-methylacridane, substituted-9-benzylidene-10-mrthylacridane, N-methylacridane or substituted N-methylacridane, 9-benzylacridane, substituted-9-benzylacridane, 9-benzyl-N-methylacridane, substituted-9-benzyl-N-methylacridane, N-alkylacridane-9-carboxylic acid or an ester or thioester thereof; indole-3-acetic acid or an ester or thioester thereof; N-methylindole-3-acetic acid oran ester thereof; phenyl or substituted phenyl-2-(6′-hydroxy-2-benzothiazolyl-.DELTA..sup.2-thiazoline-4-carboxylate, methyl 2-(6′-hydroxy-2′-benzothiazolyl)-.DELTA..sup.2-thiazoline-4-carboxylate, 2-(6′-hydroxy-2′-benzothiazolyl)-.DELTA..sup.2-thiazoline acetic acid or an ester thereof; 2-(4′-hydroxyphenyl) thiazole-4-carboxylic acid hydrazide, 2-(6′-hydroxy-2′-benzothiazolyl) thiazole-4-carboxylic acid hydrazide, 9-acridanecarboxylic acid hydrazide, substituted 9-acridanecarboxylic acid hydrazide, N-alkyl-9-acridanecarboxylic acid hydrazide, substituted N-alkyl-9-acridanecarboxylic acid hydrazide, o-hydroxybenzoic acid hydrazide, o-aminobenzoic acid hydrazide, m-hydroxybenzoic acid hydrazide, 2-hydroxy-3-naphthoic acid hydrazide, 2-amino-3-naphthoic acid hydrazide, 1-hydroxy-2-anthroic acid hydrazide, D-luciferin-O-sulfate, D-luciferin-O-phosphate, luciferins isolated from Pholas dactius, the firefly Photinus pyrali or Cypridina, and mixtures thereof.

7. The system of claim 5, wherein the homogenizing agent anionic surfactant, nonionic surfactant, protein, carbohydrate,natural polymer, or assynthetic polymer.

8. The nonionic surfactant of claim 7, wherein said nonionic surfactant is selected from glycerol, propylene glycol, Tween 20, Tween 40, Tween 60, Tween 80, Tween 85, Triton X-100, Triton X-100 (reduced), Triton N-101, Triton N-101 (reduced), Triton X-114, Triton X-114 (reduced), Triton X-405, Triton X-405 (reduced), and Brij 35.

9. The ionic surfactant of claim 7, wherein said ionic surfactant is selected from lauryl sulfate, domiphen bromide, cetyltrimethyl ammonium bromide, cetyltrimethyl ammonium chloride, cetyldimethylethyl ammonium bromide (CTAB), protein, polymer, carbohydrate, inorganic pyrophosphates, ethylenediaminetetraacetic acid (EDTA), diethylenetriaminepentaacetic acid, and ethylene-bis(oxyethylenenitrilo) tetraacetic acid.

10. The protein of claim 7, wherein said protein is selected from gelatin, bacitracin, BSA, KLH, HSA, and trypsin inhibitor.

11. The polymer of claim 7, wherein said polymer is selected from polyvinyl alcohol and polylysine.

12. The carbohydrate of claim 7, wherein said carbohydrate is selected from the group consisting of hydroxyethyl cellulose, hydroxypropyl cellulose, and carboxymethyl cellulose.

13. The system of claim 5, wherein Component A further comprises:(a) from about 0.01% to about 10% based on weight, of the chemiluminescent compound, (b) from about 0.01% to about 10% based on weight, of the enhancer, and (c) from about 0.01% to about 30% based on weight, of the homogenizer.

14. The system of claim 5, wherein component A further comprises of a solubilizing agent.

15. The system of claim 14, wherein said solubilizing agent comprises from about 0.05% to about 10%, by volume, based on the total volume, of component A.

16. The system of claim 14, wherein the solubilizing agent is selected from the group consisting of dimethyl formamide, dimethyl sulfoxide, Tetrahydrofuran, dioxane, alcohols and mixtures there of.

17. The system of claim 5, wherein the enhancer is selected from the group consisting of halogenated phenols, wherein said halogenated phenol is selected from p-iodophenol, p-bromophenol, p-chlorophenol, 4-bromo-2-chlorophenol, 3,4-dichlorophenol, alkylated phenols, 4-methylphenol, 4-tert-butylphenol, 3-(4-hydroxyphenyl) propionate, 4-benzylphenol, 4-(2′,4′-dinitrostyryl) phenol, 2,4-dichlorophenol, p-hydroxycinnamic acid, p-fluorocinnamic acid, p-nitroicinnamic acid, p-aminocinnamic acid, m-hydroxycinnamic acid, o-hydroxycinnamic acid, 4-phenoxyphenol, 4-(4-hydroxyphenoxy) phenol, p-phenylphenol, 2-chloro-4-phenylphenol, 4′-(4′-hydroxyphenyl) benzophenone, 4-(phenylazo) phenol, 4-(2′-carboxyphenylaza) phenol, 1,6-dibromonaphtho-2-ol, 1-bromonaphtho-2-ol, 2-naphthol, 6-bromonaphth-2-ol, 6-hydroxybenzothiazole, 2-amino-6-hydroxybenzothiazole, 2,6-dihydroxybenzothiazole, 2-cyano-6-hydroxybenzothiazole, dehydroluciferin, firefly luciferin, phenolindophenol, 2,6-dichlorophenolindophenol, 2,6-dichlorophenol-o-cresol, phenolindoaniline, N-alkylphenoxazine or substituted N-alkylphenoxazine, N-alkylphenothiazine or substituted N-alkylphenothiazine, N-alkylpyrimidyl-phenoxazine or substituted N-alkylpyrimidylphenoxazine, N-alkylpyridylphenoxazine, 2-hydroxy-9-fluorenone or substituted 2-hydroxy-9-fluorenone, 6-hydroxybenzoxazole or substituted 6-hydroxybenzoxazole.

18. The system of claim 5, wherein the enhancer is selected from a protected enhancer, phenolic phosphates, p-phenylene diamine, tetramethyl benzidine, fluorescein, and 5-(n-tetradecanyl) amino fluorescein.

19. The system of claim 5, wherein the chemiluminescent compound is selected from the group consisting of luminol, isoluminol, phenyl-10-methylacridane-9-carboxylate, 2,4,6-trichlorophenyl-1-O-methylacridane-9-carboxylate, acridane, pyrogallol, phloroglucinol, resorcinol, and mixtures thereof.

20. The system of claim 5, wherein the oxidizing agent is selected from the group consisting of hydrogen peroxide, urea hydrogen peroxide, a perborate salt and mixtures thereof.

21. A method for performing chemiluminescence measurements, comprising the steps of:
(a) mixing the component A and the component B of claim 5, (b) providing a detection probe which reacts with the chemiluminescent substrate to emit detectable light, and
(c) detecting the relative light emitted by the interaction of the detection probe with the chemiluminescent substrate.

22. The method of claim 21, wherein the probe comprises an enzyme, haemoglobin, protohemin, cytochrome C, or a related biomimetic model.

23. The method of claim 22, wherein the enzyme is selected from the group consisting of horseradish peroxidase, soyabean peroxidase, xanthine oxidase, catalase, laccase, and mixtures thereof.

24. A method of performing an immunological assay of an analyte concentration in a test sample using the system of claim 5, comprising the steps of:
(a) mixing component A and component B to form a premixed solution;
(b) providing an analyte in a test sample and in a standard of known analyte concentration;
(c) providing an antibody conjugated to a detection probe which reacts with the chemiluminescent substrate to emit detectable light, wherein said antibody binds to the analyte in the test sample and in the standard;
(d) contacting said antibody separately with the analyte in the test sample and in the standard to allow binding of the antibody to the analyte in the test sample and in the standard, forming a bound test sample and a bound standard;
(e) contacting the premixed solution separately with the bound test sample and the bound standard, allowing the detection probe to react with the chemiluminescent substrate to emit detectable light; and
(f) detecting the relative light emitted by the interaction of the detection probe with the chemiluminescent substrate in the bound test sample and the bound standard; and
(g) calculating the analyte concentration in the test sample based on the relative amounts of light detected in the bound test sample and the bound standard in step (f).

25. The method of claim 24, wherein the light emitted in the bound test sample and in the bound standard shows a proportional decay with time.

26. The method of claim 25, wherein the decay of the light emitted does not effect the concentration of the analyte measured over the entire analyte measurement range of the immunoassay.

27. The method of claim 22, wherein the analyte may be measured over a time period of from about one (1) to twenty (2) minutes after contacting the premixed solution with the bound test sample and the bound standard.
